(19) United States
(12) Patent Application Publication
(10) Pub. No.: US 2021/0128419 A1
(43) Pub. Date: May 6, 2021

(54) DRIP-FREE CLEANSING MASK WITH ENHANCED ACTIVE DEPOSITION

(71) Applicant: L'OREAL, Paris (FR)

(72) Inventors: Nicholas David STEBBINS, Clark, NJ (US); Ryuji HARA, Westfield, NJ (US); Susan HALPERN CHIRCH, Basking Ridge, NJ (US)

(73) Assignee: L'OREAL, Paris (FR)

(21) Appl. No.: 16/668,494

(22) Filed: Oct. 30, 2019

(51) Int. Cl. A61K 8/02; A61Q 19/10; A61K 8/87; A61K 8/85; A61K 8/81; A61K 8/46; A61K 8/44; A61K 8/368

(52) U.S. Cl. CPC A61K 8/0212; A61Q 19/10; A61K 8/87; A61K 8/368; A61K 8/8152; A61K 8/466; A61K 8/44; A61K 8/85

(57) ABSTRACT

Cleansing mask composition are provided in the present disclosure. The cleansing mask compositions includes a) from about 0.4 to 9 wt. % of a first thickener chosen from non-acrylate base rheology modifier; b) from about 0.2 to about 2 wt. % of a second thickener chosen from acrylic- or acrylate based thickeners; c) from about 2 to 25 wt. % of at least one surfactant; d) a skin care active ingredient; and wherein the and is drip free once applied on the skin.

### FIELD OF THE DISCLOSURE

The present disclosure relates to a cleansing mask composition. More particularly, the disclosure relates to a drip-free cleansing mask with enhanced active composition.

### BACKGROUND

Cosmetic cleansing and shampoo formulations are valued by a wide range of consumers. They often contain sulfate-based surfactants which facilitate the cleansing process by decreasing: the surface tension of water and thus allowing water to adhere to the dirt on the skin or hair. However, these products, due to the presence of surfactant in the composition, have a tendency of making the skin or hair too dry and, therefore, consumers prefer to use products that don't contain sulfate-base surfactants. But, sulfate-free cleansers are difficult to thicken sufficiently to afford the good sensory properties. Such cleansers do not remain on the hair or skin during and/or after application, and can drip and run into the user's eyes, mouth, ears or nasal passages. This lends to an unpleasant consumer experience. Currently two approaches are employed to thicken sulfate-free cleanser formulas. One involves using high levels of non-sulfate surfactants to benefit from the self-assembling properties of such ingredients. This approach is most common, but it is also costly. The second approach involves using high levels of rheology modifiers; however, these components can adversely impact the properties of the composition by decreasing the ease of distribution of the composition.

Therefore, it is an object of the present disclosure to create a cleansing product that provides not only an aesthetically appealing attribute upon application, but also advantageously remains in place on the keratinous surface to which it is applied so that it does not drip or run. As mentioned before, the cleansing compositions are easy to apply, drip free, and are also unique in their ability to have a good deposition of skin active ingredients.

### SUMMARY OF THE DISCLOSURE

The instant disclosure relates to cleansing masks composition comprising:
a) from about 0.4 to 9 wt. % of a first thickener chosen from non-acrylate based rheology modifier;
b) from about 0.2 to about 2 wt. % of a second thickener chosen from acrylic- or acrylate based thickeners;
c) from about 2 to 25 wt. % of at least one surfactant;
d) a skin care active ingredient; and
   wherein the composition has a loss modulus absolute value (G″) and a storage modulus absolute value (G′);
   wherein the weight percentages are based on the total weight of the cleansing mask composition.

In one or more embodiments, the loss modulus absolute value (G″) is less than three times the storage modulus absolute value (G′). In some embodiments, the composition spread easily on the skin. In some embodiments, the composition is drip-free.

In one or more embodiments, the first thickener is chosen from steareth-100/PEG-136/HDI copolymer, PEG-240/HDI copolymer bis-decyltetradeceth-20 ether, Disteareth-100 IPDI (and) Steareth-100, PEG-120 methyl glucose trioleate, and combinations thereof.

In one or more embodiments, the second thickener is chosen Acrylates/C10-30 Alkyl Acrylates, Ammonium Polyacryloyldimethyl Taurate, Ammonium Acryloyldimthyltaurate/Vp Copolymer, Sodium Acryloyldimthyltaurate/Vp Copolymer and combinations thereof.

In some embodiments, the first thickener is present from about 0.4 to about 2 wt. % based on the total weight of the cleansing mask composition.

In one or more embodiments, the second thickener is present from about 0.2 to about 2 wt. % based on the total weight of the cleansing mask composition.

In some embodiments, the at least one surfactant is chosen from anionic surfactants, amphoteric surfactants and combinations thereof. In one or more embodiments, the at least one surfactant is present from about 5 to about 12 wt. % based on the total weight of the cleansing mask composition.

In some embodiments, the skin care active ingredient is chosen from salicylic acid, alpha hydroxy acid, ceramides, ascorbic acid, antioxidants, vitamins, and combinations thereof. In some embodiments, the skin care active ingredient is deposited onto the skin. In one embodiment, the skin care active ingredient is salicylic acid.

Another aspect of the instant disclosure can include:
a) From about 0.5 to about 8 wt % of a first thickener chosen from steareth-100/PEG-136/HDI copolymer, PEG-240/HDI copolymer bis-decyltetradeceth-20 ether, Disteareth-100 IPDI (and) Steareth-100, PEG-120 methyl glucose trioleate, and combinations thereof;
b) From about 0.2 to about 2 wt % of a second thickener chosen from Acrylates/C10-30 Alkyl Acrylates, Ammonium Polyacryloyldimethyl Taurate, Ammonium Acryloyldimthyltaurate/Vp Copolymer, Sodium Acryloyldimthyltaurate/Vp Copolymer and combinations thereof.
c) From about 2 to 25 wt. % of at least one surfactant;
d) From 0.05 to 15 wt. % of a skin care active ingredients, and wherein the composition has a loss modulus absolute value (G″) and a storage modulus absolute value (G′);
   wherein the loss modulus absolute value (G″) is less than three times the storage modulus absolute value (G′);
   wherein the composition is drip free once applied on the skin;
   wherein the weight percentages are based on the total weight of the cleansing mask composition.

Another aspect of the instant disclosure can include a method of cleansing skin, comprising applying to skin the composition of claim 1.

The cleansing mask compositions of the instant disclosure provide unexpected aesthetics appealing attribute upon applications but also advantageously remains in place on the keratinous surface to which it is applied so that it does not drip or run.

It is to be understood that both the foregoing general description and the following detailed description are exemplary and explanatory only, and are not restrictive of the invention.

### BRIEF DESCRIPTION OF THE DRAWINGS

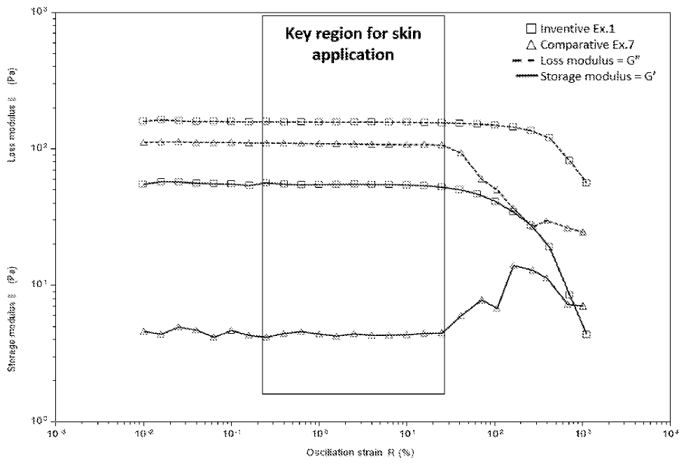

FIG. 1 is a graph showing: The measurement of the loss modulus (G″) and the storage modulus (G′) of compositions:
The loss modulus (G″) is represented with dashed lines.
The storage modulus (G′) is represented with solid lines.

The relevant % of the oscillation strain region is represented by a box.

It should be understood that the various aspects of the present disclosure are not limited to the arrangements and instrumentality shown in the FIGURES.

### DETAILED DESCRIPTION OF THE DISCLOSURE

The instant disclosure relates to compositions for cleansing the skin.

The cleansing mask compositions of the instant disclosure, in their broadest sense, typically include the following:
a) from about 0.4 to 9 wt. % of a first thickener chosen from non-acrylate base rheology modifier;
b) from about 0.2 to about 2 wt. % of a second thickener chosen from acrylic- or acrylate based thickeners;
c) from about 2 to 25 wt. % of at least one surfactant;
d) a skin care active ingredient; and
   wherein the composition has a loss modulus absolute value (G″) and a storage modulus absolute value (G′);
   wherein the weight percentages are based on the total weight of the cleansing mask composition.

The cleansing masks compositions of the instant disclosure exhibit a surprisingly drip free behavior once applied on the skin.

Without being bound by theory, it is believed that the specific combination describes in the present disclosure participates in the no dripping behavior as well as in the deposition of the skin active ingredients on the skin. Additionally, without wishing to be bound by theory, it is believed that the deposition of the salicylic acid helps oily skin and/or acne.

As used herein, the term “deposition” means that the skin active ingredient is building up onto the skin after application, and remains after rinsing away the product.

As used herein, the term “no drip” or “drip free” means that the composition once applied on the skin does not run off the surface of the skin where the cleansing mask is applied.

As used herein, the term “essentially sulfate free” means that, while it is preferable that no sulfate is present in the compositions of the invention, it is possible to have very small amounts of sulfate in the compositions, provided that these amounts do not materially affect the advantageous properties of the composition. In particular, “essentially sulfate-free anionic surfactant” means that no traditional sulfate-based anionic surfactants are used in the composition and that the surfactant does not contribute sulfate to the composition. Most preferably, the compositions contain no sulfate. To the extent any sulfate is present in the compositions, it is present at an amount of less than about 2.0% by weight, typically less than about 1.5% by weight, typically less than about 1.0% by weight, typically less than about 0.5% by weight, more typically less than about 0.1% by weight, based on the total weight of the composition. To the extent present, the sulfates in such compositions are typically contributed by components other than the anionic surfactant.

Thickeners

Non-Acrylates Based Rheology Modifier

The composition includes a non-acrylate based rheology modifier. The non-acrylates based rheology modifier exhibits a unique rheology in which the storage modulus and loss modulus are inverted relative to a traditional oil-in-water emulsion, i.e. the loss modulus is greater than the storage modulus. In compositions exhibiting both storage and loss moduli, the storage modulus measures elasticity (i.e., stored deformation energy) and partly describes the solid-like behavior and the loss modulus measures viscosity (i.e., deformation energy lost during flow) and partly describes the fluid-like behavior. Systems in which the loss modulus greater than the storage modulus exhibit fluid-like behavior. In an embodiment, the composition exhibits an inverted storage and loss moduli, which do not cross during storage or use, allowing the composition to exhibit fluid-like behavior at essentially all times.

The rheology modifiers are typically in the form of an aqueous gel. Although the gel is solid in appearance the higher loss modulus allows the gel to exhibit fluid like behavior and recovery from mechanical stress.

In various embodiments, the rheology modifiers may be
nonionic associative polyurethane polyethers:

Preferably, the nonionic associative polyurethane polyethers comprise at least two lipophilic hydrocarbon-based chains containing from 6 to 30 carbon atoms which are separated by a hydrophilic block, it being possible for the hydrocarbon-based chains to be side chains or chains at the end of the hydrophilic block. In particular, it is possible for one or more side chains to be envisaged. In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer having a hydrophilic central block) or distributed both at the ends and in the chain (for example, multiblock copolymer). These same polymers may also be graft polymers or star polymers.

The nonionic fatty-chain polyurethane polyethers may be triblock copolymers, the hydrophilic block of which is a polyoxyethylene chain comprising from 10 to 500 and preferably from 10 to 300 oxyethylene groups.

In a first variant, use may be made of a polyurethane polyether derived from the polycondensation of:

(i) a polyethylene glycol comprising from 120 to 250 mol of ethylene oxide,

(ii) a polyoxyethylenated monoalcohol comprising from 14 to 24 carbon atoms (especially decyltetradecyl alcohol or stearyl alcohol) comprising from 15 to 120 mol of ethylene oxide (especially from 20 to 100 OE), and

(iii) a diisocyanate containing from 6 to 20 carbon atoms, chosen especially from methylenebis(4-cyclohexyl isocyanate) (SMDI) and hexamethylene diisocyanate (HDI), and preferably HDI.

Such polyurethane polyethers are, for example:
the polycondensate of polyethylene glycol containing 136 mol of ethylene oxide, stearyl alcohol polyoxyethylenated with 100 mol of ethylene oxide and hexamethylene diisocyanate (HDI) especially with a weight-average molecular weight (Mw) of 30000 or 40000 (INCI name: PEG-136/steareth-100/HDI copolymer), such as the product sold under the name Rheoluxe 811 or Nuvis FX 1100 (formerly Rheolate® FX 1100) by the company Elementis.

In a second variant, use may be made of a polyurethane polyether derived from the polycondensation of:

(i) a polyethylene glycol comprising from 130 to 200 mol of ethylene oxide,

(ii) a monoalcohol comprising from 8 to 22 carbon atoms, especially decyl alcohol or stearyl alcohol, and

(iii) a diisocyanate containing from 6 to 20 carbon atoms, chosen especially from methylenebis(4-cyclohexyl isocyanate) (SMDI) and hexamethylene diisocyanate (HDI), and preferably SMDI.

Such polyurethane polyethers are, for example:
a polycondensate of polyethylene glycol containing 150 or 180 mol of ethylene oxide, stearyl alcohol and methylenebis(4-cyclohexyl isocyanate) (SMDI) (INCI name: PEG-150/stearyl alcohol/SMDI copolymer), such as the product sold at 15% by weight in a matrix of maltodextrin (4%) and water (81%) under the name Aculyn 46 by the company The Dow Chemical Company;
a polycondensate of polyethylene glycol containing 150 or 180 mol of ethylene oxide, decyl alcohol and methylenebis(4-cyclohexyl isocyanate) (SMDI) (INCI name: PEG-150/decyl alcohol/SMDI copolymer), such as the product sold at 35% by weight in a mixture of propylene glycol (39%) and water (26%) under the name Aculyn 44 Polymer by the company The Dow Chemical Company.

In a third variant, use may be made of a polycondensate of polyethylene glycol containing 200 mol of ethylene oxide, ether of polyethylene glycol containing 10 mol of ethylene oxide and/or of methylglucose, ether of polyethylene glycol containing 6 mol of ethylene oxide and of tridecyl alcohol, hexamethylene diisocyanate, and containing C16-C20 alcohol end groups, such as the product sold under the name Avalure® Flex 6 Polymer by the company Lubrizol (INCI name: Polyurethane-62).

Use is preferably made of the polyurethane polyethers described previously as examples.

The composition according to the disclosure may comprise a mixture of associative polymers as described previously. In some embodiments, the associative polymer is selected from the group consisting of steareth-100/PEG-136/HDI copolymer, PEG-240/HDI copolymer bis-decyltetradeceth-20 ether, Disteareth-100 IPDI (and) Steareth-100, PEG-240/HDI copolymer bis-decyltetradeceth-20 ether, PEG-120 methyl glucose trioleate, and combinations thereof. In some embodiments, two or more rheology modifiers are present.

Although these thickeners are given as an example, it will be appreciated that other thickeners compatible with cosmetic applications known in the art may be used.

The rheology modifiers may be present in the composition in an amount from about 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8 to about 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, or 10 wt. %, by weight percentages based on the total weight of the cleansing mask composition.

Although these thickeners are given as an example, it will be appreciated that other thickeners compatible with cosmetic applications known in the art may be used.

The first thickener may be present in the composition in an amount from about 0.4, 0.6, 0.8, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8 to about 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, or 10 wt. %, by weight percentages based on the total weight of the cleansing mask composition.

Acrylic- or Acrylate Based Thickeners

The second thickeners suitable for the instant disclosure are selected from polymers. According to the instant disclosure, the suitable thickener polymers of the instant disclosure could be selected from acrylic- or acrylate based polymers.

The polymers are pre-neutralized and preferably selected from taurate polymers. Such polymers comprise an ionic monomer portion, 2-acrylamido-2-methylpropane sulfonic acid (AMPS), as well as a further, less polar monomer portion (vinylpyrrolidone or beheneth-25 methacrylate). These polymers are used as thickeners.

Examples of taurate polymers are Acrylates/Vinyl Isodecanoate Crosspolymer (Stabylen 30 from 3V), Acrylates/C10-30 Alkyl Acrylate Crosspolymer (Pemulen TR1 and TR2), Carbomers (Aqua SF-1), Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), Non-ionic thickener, (Aculyn 46 from Rohm and Haas), or mixtures thereof.

Anionic polymers may be polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself.

The anionic polymers may comprise at least one hydrophilic unit of olefinic unsaturated carboxylic acid type, and at least one hydrophobic unit exclusively of (C10-C30)alkyl ester of unsaturated carboxylic acid type.

Anionic polymers useful herein include, for example: Polyacrylic acid; Polymethacrylic acid; Carboxyvinylpolymer; acrylate copolymers such as Acrylate/C 10-30 alkyl acrylate crosspolymer, Acrylic acid/vinyl ester copolymer/AcrylatesNinyl Isodecanoate crosspolymer, Acrylates/Palmeth-25 Acrylate copolymer, Acrylate/Steareth-20 Itaconate copolymer, and Acrylate/Celeth-20 Itaconate copolymer; sulfonate polymers such as Polysulfonic acid, Sodium Polystyrene Sulfonate supplied from Akzo Nobel under the tradename FLEXAN II, copolymers of methacrylic acid and acrylamidomethylpropane sulfonic acid, and copolymers of acrylic acid and acrylamidomethylpropane sulfonic acid; carboxymethycellulose; carboxy guar gum; copolymers of ethylene and maleic acid; and acrylate silicone polymer. In some instances, the anionic polymers include, for example, Carbomer supplied from Noveon under the tradename CARBOPOL 981 and CARBOPOL 980; Acrylates/C10-30 Alkyl Acrylate Crosspolymer having tradenames Pemulen TR-1, PEMULEN TR-2, CARBOPOL 1342, CARBOPOL 1382, and CARBOPOL ETD 2020, all available from Noveon; sodium carboxymethylcellulose supplied from Hercules as CMC series; and Acrylate copolymer having a tradename Capigel supplied from Seppic; acrylates copolymer having the tradename CARBOPOL Aqua SF-1 and available from Lubrizol as an aqueous dispersion, and acrylates crosspolymer-4 having the tradename CARBOPOL Aqua SF-2 and available from Lubrizol as an aqueous dispersion.

In an embodiment, the anionic polymer of the invention is carbomer which may be commercially available from the supplier Lubrizol under the tradename of CARBOPOL 980. Exemplary of non-ionic polymers could be as follows:

(i) hydroxyethylcellulose, for instance the product NATROSOL 250 HHR PC or NATROSOL 250 HHR CS sold by the company Ashland;

(ii) celluloses modified with groups comprising at least one fatty chain; examples that may be mentioned include:
hydroxyethylcelluloses modified with groups comprising at least one fatty chain, such as alkyl, arylalkyl or alkylaryl groups, or mixtures thereof, and in which the alkyl groups are preferably C8-C22, for instance the product NATROSOL Plus Grade 330 CS (C16 alkyls) sold by the company Ashland, or the product BERMOCOLL EHM 100 sold by the company AkzoNobel; methyl hydroxyethylcellulose; methyl ethyl hydroxyethylcellulose, known as the product STRUCTURE CEL 8000 M sold by the company AkzoNobel; or hydroxypropyl cellulose, known as the product KLUCEL MF PHARM HYDROXYPROPYLCELLULOSE sold by the company Ashland;
hydroxyethylcelluloses modified with alkylphenyl polyalkylene glycol ether groups, such as the product Amercell Polymer HM-1500 (nonylphenyl polyethylene glycol (15) ether) sold by the company Amerchol; or

(iii) hydroxypropyl guars such as hydroxypropyl guar sold by as the product JAGUAR HP 105 by the company Rhodia and hydroxypropyl guars modified with groups comprising at least one fatty chain, such as the product Esaflor HM 22 (C22 alkyl chain) sold by the company Lamberti, and the products RE210-18 (C14 alkyl chain) and RE205-1 (C20 alkyl chain) sold by the company Rhodia

In some embodiments, the second thickener may be a hydrophilic acrylic polymer. In some embodiments, the second thickener is selected from the group consisting of Acrylates/C10-30 Alkyl Acrylates, Ammonium Polyacryloyldimethyl Taurate, Ammonium Acryloyldimthyltaurate/Vp Copolymer, Sodium Acryloyldimthyltaurate/Vp Copolymer and combinations thereof.

Accordingly, the amount of the second thickener in the composition according to the instant disclosure may be from about 0.4, 0.5, 0.6, 0.7, 0.8, 0.9, 1.0, 1.1 to about 1.2, 1.3, 1.4, 1.5, 1.6, 1.7, 1.8, 1.9 or 2.0 wt. %, by weight percentages based on the total weight of the cleansing mask composition.

Surfactants

The surfactant can result from a combination of different surfactants, including anionic, nonionic, and amphoteric (zwitterionic) surfactants. Anionic surfactants carry a negative charge on the polar head group. These surfactants are typically used for their detergency properties. They are highly effective at removing dirt and oil from the hair and scalp. Nonionic surfactants are those that have no (or very little) residual electric charge. These surfactants can perform a variety of functions, such as emulsion stabilization, mild detergency and viscosity modification. Amphoteric (zwitterionic) surfactants are dual-charged (have both a positive and negative charge on the molecule). Many amphoteric surfactants display pH-dependent charge behavior, having one charge at a lower pH and the opposite charge at a higher pH. These types of surfactants tend to be mild both to skin and hair. They can also provide foam-boosting properties in combination with anionic surfactants, which enhances lather.

The surfactant of the instant disclosure includes: (i) one or anionic surfactants; and (ii) one or more amphoteric surfactants. The surfactant system may also optionally include: (iii) additional miscellaneous nonionic surfactants.

The total amount of surfactants in the instant disclosure may be present in the ranges from about 2 to about 25 wt. %, by weight percentages based on the total weight of the cleansing mask composition. On some embodiments, the total amounts of surfactants maybe from about 4 to about 12 wt. %, by weight percentages based on the total weight of the cleansing mask composition.

The total amount of surfactants in the instant disclosure may be present from about 2, 3, 4, 5, 6, 7, 8, 9, 10, 11, 12, 13, 14 to about 14, 15, 16, 17, 18, 19, 20, 21, 22, 23, 24, or 25 wt. %. by weight percentages based on the total weight of the cleansing mask composition.

Useful but non-limiting examples of surfactants that may be used in the instant disclosure are provided below.

(i) Non-Sulfate Anionic Surfactants

Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof. Non-limiting examples of useful non-sulfate anionic surfactants are provided below.

(i-a) Alkyl Sulfonates

Useful alkyl sulfonates include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (III) is particularly useful.

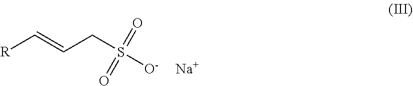

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (III) but the cation may be an alkali metal ion such as sodium or potassium, ammonium ions, or alkanolammonium ions such as monoethanolammonium or triethanolammonium ions. In some instances, the alkyl sulfonate(s) are selected from C8-C16 alkyl benzene sulfonates, C10-C20 paraffin sulfonates, C10-C24 olefin sulfonates, salts thereof, and mixtures thereof. C10-C24 olefin sulfonates are particularly preferred. A non-limiting but particularly useful example of a C10-C24 olefin sulfonate that can be used in the instant compositions is sodium C14-16 olefin sulfonate.

(i-b) Alkyl Sulfosuccinates

Non-limiting examples of useful sulfosuccinates include those of formula (IV):

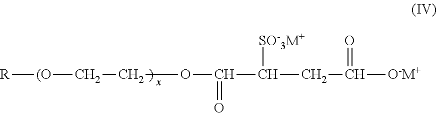

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

(i-c) Alkyl Sulfoacetates

Non-limiting examples of alkyl sulfacetates includes, for example, alkyl sulfoacetates such as C4-C18 fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

(i-d) Acyl Isethionates

Non-limiting examples of useful acyl isethionates include those of formula (V) and (VI):

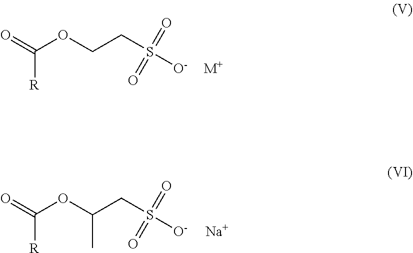

wherein R, R1, R2 and R3 are each independently selected from H or an alkyl chain having 1-24 carbon atoms, said chain being saturated or unsaturated, linear or branched, and X is COO— or SO3-. Sodium is shown as the cation in formula (VI) but the cation for both formula (V) and formula (VI) may be an alkali metal ion such as sodium or potassium, ammonium ions, or alkanolammonium ions such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate.

(i-e) Alkoxylated Monoacids

Non-limiting examples of alkoxylated monoacids include compounds corresponding to formula (VII):

RO[CH2O]u[(CH2)xCH(R′)(CH2)y(CH2)zO]v[CH2CH2O]wCH2COOH (VII)

wherein:
R is a hydrocarbon radical containing from about 6 to about 40 carbon atoms;
u, v and w, independently of one another, represent numbers of from 0 to 60;
x, y and z, independently of one another, represent numbers of from 0 to 13;
R′ represents hydrogen, alkyl, and
the sum of x+y+z>0;

Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C6-40 alkyl or alkenyl group or a C1-40 alkyl phenyl group, more typically a C8-22 alkyl or alkenyl group or a C4-18 alkyl phenyl group, and even more typically a C12-18 alkyl group or alkenyl group or a C6-16 alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

(i-f) Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of useful acyl amino acids include those of formula (VIII):

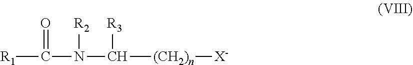

wherein R, R1, R2 and R3 are each independently selected from H or an alkyl chain having 1-24 carbon atoms, said chain being saturated or unsaturated, linear or branched, and X is COO— or SO3-.

Acyl Taurates: Non-limiting examples of acyl taurates include those of formula (IX):

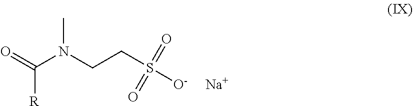

wherein R, R1, R2 and R3 are each independently selected from H or an alkyl chain having 1-24 carbon atoms, or from 6-20 carbon atoms, or from 8 to 16 carbon atoms, said chain being saturated or unsaturated, linear or branched, and X is COO— or SO3-. Non-limiting examples of acyl taurate salts include sodium cocoyl taurate and sodium methyl cocoyl taurate.

Acyl Glycinates: Non-limiting examples of useful acyl glycinates include those of formula (X):

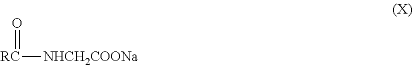

wherein R is an alkyl chain of 8 to 16 carbon atoms. Sodium is shown as the cation in the above formula (X) but the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

Acyl Glutamates: Non-limiting examples of useful acyl glutamates include those of formula (XI):

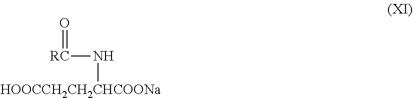

wherein R is an alkyl chain of 8 to 16 carbon atoms. Sodium is shown as the cation in the above formula (XI) but the cation may be an alkali metal ion such as sodium or potassium, ammonium ions, or alkanolammonium ions such as monoethanolammonium or triethanolammonium ions. Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium oleoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

Acyl Sarcosinates: Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

(ii) Alkyl Polyglucosides

Alkyl polyglucosides are a class of nonionic surfactants. The total amount of alkyl polyglucoside(s) in the cleansing compositions may vary but is typically from about 2 to about 25 wt. %, based on the total weight of the cleansing composition. In some instance, the total amount of alkyl polyglucoside(s) in the cleansing composition is from about 2 to about 20 wt. %, from about 2 to about 15 wt. %, from about 2 to about 10 wt. %, from about 5 to about 25 wt. %, from about 5 to about 20 wt. %, from about 5 to about 15 wt. %, or from about 5 to about 10 wt. %, based on the total weight of the cleansing composition.

Useful polyglucosides include alkyl polyglucosides having the following formula (XII):

R1-O—(R2O)n-Z(x) (XII)

wherein R1 is an alkyl group having 8-18 carbon atoms;
R2 is an ethylene or propylene group;
Z is a saccharide group with 5 to 6 carbon atoms;
n is an integer from 0 to 10; and
x is an integer from 1 to 5.

Useful alkyl poly glucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

(iii) Amphoteric Surfactants

Useful amphoteric surfactants include betaines, alkyl sultaines, alkyl amphoacetates, alkyl amphopropionates, and mixtures thereof. Non-limiting examples of useful amphoteric surfactants are provided below.

(iii-a) Betaines

Useful betaines include those of the following formulae (XIIIa-XIIId):

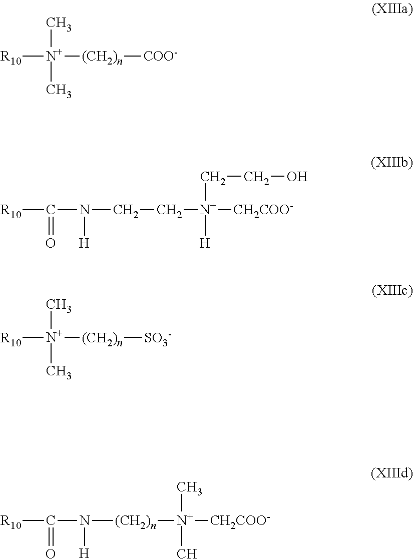

wherein R10 is an alkyl group having 8-18 carbon atoms; and n is an integer from 1 to 3.

Particularly useful betaines include, for example, coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, cocamidopropyl betaine, behenyl betaine, capryl/capramidopropyl betaine, and lauryl betaine, and mixtures thereof. Particularly preferred betaines include coco betaine and cocamidopropyl betaine.

(iii-b) Alkyl Sulltaines

Non-limiting examples of alkyl sultaines include hydroxyl sultaines of formula (XIV)

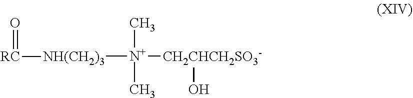

wherein R is an alkyl group having 8-18 carbon atoms. More specific examples include, but are not limited to cocamidopropyl hydroxysultaine, lauryl hydroxysultaine, and a mixture thereof.

(iii-c) Alkyl Amphoacetates and Alkyl Amphodiacetates

Useful alkyl amphoacetates and alkyl amphodiacetates include those of Formula (XV) and (XVI):

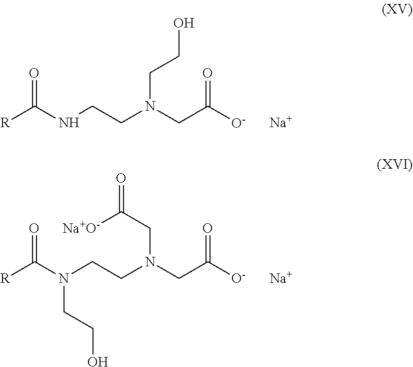

wherein R is an alkyl group having 8-18 carbon atoms. Sodium is shown as the cation in the above formulae above but the cation may be an alkali metal ion such as sodium or potassium, ammonium ions, or alkanolammonium ions such as monoethanolammonium or triethanolammonium ions. A more specific, but non-limiting example, is sodium lauroamphoacetate.

(iii-d) Alkyl Amphopropionates

Non-limiting examples of alkyl amphopropionates include cocoamphopropionate, cornamphopropionatecaprylamphopropionate, cornamphopropionate, caproamphopropionate, oleoamphopropionate, isostearoamphopropionate, stearoamphopropionate, lauroamphopropionate, salts thereof, and a mixture thereof.

(iv) Miscellaneous Nonionic Surfactants

The cleansing compositions may optionally include one or more miscellaneous nonionic surfactants, i.e., one or more nonionic surfactants in addition to the alkyl polyglucosides and the amide surfactants discussed above.

The nonionic surfactant(s) can be, for example, selected from alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C6-C24)alkylpolyglycosides; N—(C6-C24)alkylglucamine derivatives, amine oxides such as (C10-C14)alkylamine oxides or N—(C10-C14)acylaminopropylmorpholine oxides; and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

Skin Care Active Ingredients

Organic Acid

The composition according to the instant disclosure comprises at least one organic acid as cosmetic active ingredient.

Many organic acids can be used as cosmetic active ingredient to bring specific efficacy for example anti-Acne effect and/or peeling effect.

Peeling is well-known approach to improve the appearance and/or the texture of the skin and/or of the scalp, in particular improving the radiance and the homogeneity of the complexion and/or reducing the visible and/or tactile irregularities of the skin, and in particular for improving the surface appearance of the skin, for reducing actinic lentigo, acne marks and chickenpox marks, and also for preventing, reducing or combating the signs of skin aging, and in particular for smoothing out the irregularities of the skin's texture, such as wrinkles and fine lines.

Peeling has the effect of removing a superficial part of the skin (epidermis and possibly superficial layer of the dermis).

Organic acid can be used as anti-acne and/or peeling agent is preferably selected from saturated and unsaturated monocarboxylic acids, saturated and unsaturated dicarboxylic acids, saturated and unsaturated tricarboxylic acids; α-hydroxy acids and β-hydroxy acids of monocarboxylic acids; α-hydroxy acids and β-hydroxy acids of dicarboxylic acids; α-hydroxy acids and β-hydroxy acids of tricarboxylic acids; keto acids, α-keto acids, β-keto acids of polycarboxylic acids, of polyhydroxymonocarboxylic acids, of polyhydroxydicarboxylic acids or of polyhydroxytricarboxylic acids; and (3-hydroxy-2-pentylcyclopentyl)acetic acid.

Preferred α-hydroxy acids that are exemplary include: glycolic acid, citric acid, lactic acid, tartaric acid, malic acid or mandelic acid.

Preferred β-hydroxy acids are selected from: salicyclic acid and derivatives thereof, in particular 5-n-octanoylsalicylic acid.

Those skilled in the art will be able to define the required amount of organic acid present in the composition according to the invention in order to obtain the desired effect on the skin.

The skin care active ingredient may be in an amount from about 0.05%, 0.06, 0.08, 0.1, 0.2, 0.4, 0.6, 0.8, 1.0, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7% to about 7, 7.2, 7.4, 7.6, 7.8, 8, 8.2, 8.4, 8.6, 8.8, 9, 9.2, 9.4, 9.6, 9.8, 10, 10.2, 10.4, 10.6, 10.8, 11, 11.2, 11.6, 11.8, 12, 12.2, 12.6, 12.8, 13, 13.2, 13.4, 13.6, 13.8, 14, 14.2, 14.4, 14.6, 14.8, or 15% wt., by weight percentages based on the total weight of the cleansing mask composition.

Cosmetically Acceptable Carrier

The water and or the glycols in the compositions typically form part or all of a cosmetically acceptable carrier. The cosmetically acceptable carrier can include, for example, glycerin, C1-4 alcohols, organic solvents, fatty alcohols, fatty ethers, fatty esters, polyols, glycols, vegetable oils, mineral oils, liposomes, laminar lipid materials, water, or any combinations thereof. As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

In some instances, cosmetically acceptable carriers may comprise water, a mixture of water and at least one cosmetically acceptable organic solvent, or at least one cosmetically acceptable organic solvent. Additionally, cosmetically acceptable carriers may be or may include ethanol, a glycol ether, for example, dipropylene glycol n-butyl ether, isododecane, mineral oil, propylene glycol, pentylene glycol, hexylene glycol, glycerol, and mixtures thereof.

Optional Components

In one embodiment, the composition may include optional components selected from the group consisting of actives, fragrance, preservatives, and combinations thereof. The actives are selected from the group consisting of butylated hydroxytoluene, tocopherol, tocopheropl derivatives, tocotrienol, tocotrienol derivatives, ascorbic acid, ascorbic acid derivatives, ascorbyl palmitate, vitamin E, vitamin C, and combinations thereof.

The compositions may also include any other adjuvant or additive that is usually used in the field of self-cleansing products, in particular shampoos. A person skilled in the art would know which adjuvants and/or additives to select to achieve the desired results (e.g. preservatives) without adversely affecting the properties of claimed emulsions. For example, such additives include preserving agents (e.g. phenoxyethanol, sodium benzoate, benzoic acid), consistency regulators (e.g. isopropyl alcohol), thickeners, antioxidants, fragrances, and mixtures thereof.

The above ingredients lists are only examples and not limiting.

The compositions according to the instant disclosure may be prepared according to techniques that are well known to those skilled in the art, in particular those intended for the preparation of shaving compositions.

The instant disclosure will be better understood from the examples that follow, all of which are intended for illustrative purposes only and are not meant to limit the scope of the instant disclosure in any way.

As used herein, the terms “comprising,” “having,” and “including” are used in their open, non-limiting sense.

The terms “a,” “an,” and “the” are understood to encompass the plural as well as the singular.

The expression “at least one” means one or more and thus includes individual components as well as mixtures/combinations.

The expression “one or more” as used herein includes individual components as well as mixtures/combinations.

All ranges and values disclosed herein are inclusive and combinable. For examples, any value or point described herein that falls within a range described herein can serve as a minimum or maximum value to derive a sub-range, etc.

All percentages, parts and ratios herein are based upon the total weight of the compositions of the present disclosure, unless otherwise indicated.

All ranges and values disclosed herein are inclusive and combinable. For examples, any value or point described herein that falls within a range described herein can serve as a minimum or maximum value to derive a sub-range, etc.

Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### EXAMPLES

The following examples illustrate the present invention but are not intended to limit the scope of the invention.

TABLE 1
Inventive Examples

| | | | Inventive | Inventive | Inventive | Inventive | Inventive | Inventive |
|---|---|---|---|---|---|---|---|---|
| Claims | Function | US INCI Name | Ex.1 | Ex.2 | Ex.3 | Ex.4 | Ex.5 | Ex.6 |
| a | Rheology Modifier | STEARETH-100/PEG-136/HDI | 6 | 0 | 7.5 | 6 | 0 | 0 |
| | | COPOLYMER | | | | | | |
| | | PEG-120 METHYL GLUCOSE | 0 | 1 | 0 | 0 | 0.5 | 1 |
| | | DIOLEATE | | | | | | |
| | | DISTEARETH-100 IPDI (and) | 0 | 1 | 0 | 0 | 0.5 | 1 |
| | | STEARETH-100 | | | | | | |
| b | Thickener | AMMONIUM | 1.3 | 0 | 1 | 1.4 | 0 | 0 |
| | | POLYACRYLOYLDIMETHYL | | | | | | |
| | | TAURATE | | | | | | |
| | | ACRYLATES/C10-30 ALKYL | 0 | 0.72 | 0 | 0 | 1.08 | 0.72 |
| | | ACRYLATE CROSSPOLYMER | | | | | | |
| | | AMMONIUM | 0 | 0 | 0 | 0 | 0 | 0 |
| | | ACRYLOYLDIMETHYLTAURATE/ | | | | | | |
| | | STEARETH-25 METHACRYLATE | | | | | | |
| | | CROSSPOLYMER | | | | | | |
| | | ACRYLATES/VINYL | 0 | 0 | 0 | 0 | 0 | 0 |
| | | ISODECANOATE CROSSPOLYMER | | | | | | |
| c | Surfactant | SODIUM C14-16 OLEFIN | 3.6 | 3.6 | 3.6 | 3.6 | 3.6 | 3.6 |
| | | SULFONATE | | | | | | |
| | | LAURYL BETAINE | 3.6 | 0 | 3.6 | 0 | 0 | 0 |
| | | PEG-7 GLYCERYL COCOATE | 0 | 1 | 0 | 1 | 1 | 1 |
| | | COCO-BETAINE | 0 | 2.7 | 0 | 3.6 | 2.7 | 2.7 |
| | | SODIUM LAURYL SULFOACETATE | 0 | 0 | 0 | 0 | 0 | |
| | | DISODIUM LAURETH | 0 | 0 | 0 | 0 | 0 | 0 |
| | | SULFOSUCCINATE | | | | | | |
| d | Skin Care Active | SALICYLIC ACID | 1.5 | 1.5 | 1 | 1.5 | 2 | 1.5 |
| | Carrier | GLYCOLS | 0.3 | | 0.3 | 0.3 | 0 | 0.3 |
| | | GLYCERIN | 5 | 6 | 5 | 5 | 6 | 6 |
| | | WATER/AQUA | Q.S. | Q.S. | Q.S. | Q.S. | Q.S | Q.S. |
| | preservative, | SODIUM HYDROXIDE; | 0.01-3 | 0.01-3 | 0.01-3 | 0.01-3 | 0.01-3 | 0.01-3 |
| | additives, Fragrance | PHENOXYETHANOL, CHARCOAL | | | | | | |
| | | POWDER, MENTHOL, TRISODIUM | | | | | | |
| | | ETHYLENEDIAMINE | | | | | | |
| | | DISUCCINATE, TETRA SODIUM | | | | | | |
| | | EDTA, CITRIC ACID, SODIUM | | | | | | |
| | | CITRATE | | | | | | |
| | DRIP | | NO | NO | NO | NO | NO | NO |
| | EASE OF APPLICATION | | EASY | EASY | EASY | EASY | EASY | EASY |

The Inventive Examples in Table 1 were prepared according to the procedure as follows. The solvents/carriers were mixed with water, then the thickeners(s) were added until well-dispersed. The pH was adjusted to neutralize the thickener(s) (if necessary). Actives were then added and mixed into the batch Surfactant(s) were then added and batch was stirred until uniform. The rheology modifier(s) were then added and mixed until batch was uniform. Finally, any other preservatives, additives, and fragrance were added, if present. Once well-mixed and uniform, the batch was complete.

Evaluation of the Rheology of the Composition

TABLE 2
Comparative Examples

| | | | Compar- | Compar- | Compar- | Compar- | Compar- | Compar- | Compar- | Compar- |
|---|---|---|---|---|---|---|---|---|---|---|
| | | | ative | ative | ative | ative | ative | ative | ative | ative |
| Claims | Function | US INCI Name | Ex. 1 | Ex. 2 | Ex. 3 | Ex. 4 | Ex. 5 | Ex. 6 | Ex. 7 | Ex. 8 |
| a | Rheology | STEARETH-100/PEG-136/ | 0 | 0 | 0 | 0 | 0 | 0 | 6 | 0 |
| | Modifier | HDI COPOLYMER | | | | | | | | |
| | | PEG-120 METHYL | 5 | 0 | 0 | 0 | 0 | 0 | 0 | 0 |
| | | GLUCOSE DIOLEATE | | | | | | | | |
| | | DISTEARETH-100 IPDI | 5 | 8 | 0 | 0 | 0 | 0 | 0 | 0 |
| | | (and) STEARETH-100 | | | | | | | | |
| b | Thickener | AMMONIUM | 0 | 0 | 0 | 0 | 0 | 0 | 0 | 1.3 |
| | | POLYACRYLOYL- | | | | | | | | |
| | | DIMETHYL TAURATE | | | | | | | | |
| | | ACRYLATES/C10-30 | 0 | 0 | 0 | 0.98 | 0 | 1.35 | 0 | 0 |
| | | ALKYL ACRYLATE | | | | | | | | |
| | | CROSSPOLYMER | | | | | | | | |
| | | AMMONIUM | 0 | 0 | 1.7 | 0 | 0 | 0 | 0 | 0 |
| | | ACRYLOYL- | | | | | | | | |
| | | DIMETHYLTAURATE/ | | | | | | | | |
| | | STEARETH-25 | | | | | | | | |
| | | METHACRYLATE | | | | | | | | |
| | | CROSSPOLYMER | | | | | | | | |
| | | ACRYLATES/VINYL | 0 | 0 | 0 | 0 | 1.47 | 0 | 0 | 0 |
| | | ISODECANOATE | | | | | | | | |
| | | CROSSPOLYMER | | | | | | | | |
| c | Surfactant | SODIUM C14-16 OLEFIN | 0 | 3.6 | 3.6 | 3.6 | 3.6 | 3.6 | 3.6 | 3.6 |
| | | SULFONATE | | | | | | | | |
| | | LAURYL BETAINE | 0 | 0 | 0 | 0 | 0 | | 3.6 | 3.6 |
| | | PEG-7 GLYCERYL | 1.05 | 0 | 1 | 1 | 1 | 1 | 0 | 0 |
| | | COCOATE | | | | | | | | |
| | | COCO-BETAINE | 1.5 | 3.6 | 3.6 | 0 | 2.7 | 2.7 | 0 | 0 |
| | | SODIUM LAURYL | 1.6 | 0 | 0 | 0 | 0 | 0 | 0 | 0 |
| | | SULFOACETATE | | | | | | | | |
| | | DISODIUM LAURETH | 4.05 | 0 | 0 | 0 | 0 | 0 | 0 | 0 |
| | | SULFOSUCCINATE | | | | | | | | |
| d | Skin Care | SALICYLIC ACID | 2 | 0 | 2 | 2 | 1.5 | 1.5 | 1.5 | 1.5 |
| | Active | | | | | | | | | |
| | Carrier | GLYCOLS | 0.3 | 0 | 0 | 0 | 0 | 1 | 0.5 | 0.5 |
| | | GLYCERIN | | 5 | 6 | 8 | 6 | 6 | 5 | 5 |
| | | WATER/AQUA | Q.S. | Q.S. | Q.S. | Q.S. | Q.S. | Q.S. | Q.S. | Q.S. |
| | preservative, | SODIUM HYDROXIDE; | 0.01-3 | 0.01-3 | 0.01-3 | 0.01-3 | 0.01-3 | 0.01-3 | 0.01-3 | 0.01-3 |
| | additives, | PHENOXYETHANOL, | | | | | | | | |
| | Fragrance | CHARCOAL POWDER, | | | | | | | | |
| | | MENTHOL, TRISODIUM | | | | | | | | |
| | | ETHYLENEDIAMINE | | | | | | | | |
| | | DISUCCINATE, TETRA | | | | | | | | |
| | | SODIUM EDTA, CITRIC | | | | | | | | |
| | | ACID, SODIUM CITRATE | | | | | | | | |
| | DRIP | | NO | NO | YES | YES | YES | NO | NO | YES |
| | EASE OF | | DIFFI- | DIFFI- | UN- | EASY | EASY | UN- | DIFFI- | RUNNY |
| | APPLICATION | | CULT | CULT | STABLE | | | STABLE | CULT | |

Experiments

Rheological Measurement

The rheological measurements of the compositions were conducted with a TA Instruments Discovery HR-2 Hybrid Rheometer. Experiments were performed with a 40 mm diameter parallel plate geometry with 1 mm gap, and at a controlled temperature of 25° C. throughout sample acquisition. After loading, sample was equilibrated for 1 min. Then, an amplitude sweep experiment was performed at a frequency of 1 Hz and a % oscillation strain ranging from 0.01 to 1000. Data was analyzed using TRIOS software. (See FIG. 1)

Interpretation

The loss modulus absolute value G″ for Inventive Ex.1 (squares, dashed line) was less than 3 times the storage modulus absolute value G′ for the same formula (squares, solid line). See FIG. 1. Inventive Ex.1 showed good spreading over the skin and a drip-free texture.

The loss modulus absolute value G″ for Comparative Ex. 7 (triangles, dashed line) was significantly higher than the storage modulus absolute value G′ for the same formula (triangles, solid line) See FIG. 1.

The Comparative Ex. 7 exhibited very poor spreading on skin and was not able to be uniformly applied. It is important to note that the absolute values of G′ and G″ in Pa is not a key attribute for the instant disclosure; it is the ratio between them that is important. That is, the G″ and G′ were greater for the Inventive Ex. 1 than the respective values of G″ and G′ for the Comparative Ex. 7; however, the spreading was easy and drip-free for the Inventive Ex. 1 because G″ was less than three times G′.

Note: The relevant % oscillation strain region correlating to skin care application (i.e., rubbing product on skin) for the measurements is represented in a box for clarification.

Evaluation on Skin

A known amount of composition was picked up with finger and applied to the forearm while elevated and parallel to the ground. A thick layer was applied and rubbed onto skin surface and rubbed gently in a back and forth motion for 3 seconds. During application, records were collected if the formulation remained smooth or if any unacceptable ‘pilling’ or ‘balling up’ took place. Then, any dripping was assessed by observing over a time period of a few minutes any migration of the formula downward (away from the initial application site) due to gravity.

Interpretation

Comparative Ex. 7 contained the same amount of rheology modifier (i.e. Steareth-100/PEG-136/HDI Copolymer) as Inventive Ex. 1, as well as the other ingredients, the only difference is the absence of a second thickener. When compared to Inventive Ex. 1, it was observed that when the rheology modifier was present without any other thickener, the cleansing mask didn't drip, but was too thick and couldn't be spread easily onto the skin.

Comparative Ex. 8 contained no rheology modifiers, but contained the same amount of thickener as Inventive Ex. 1 as well as the other ingredients. When compared to Inventive Ex. 1, it was observed that when the rheology modifier is not associated with the second thickener, the cleansing mask was too thin and was dripping once applied onto the skin.

According to the results described above, the association of a first thickener such as a non-acrylate based rheology modifier with a thickener such as an acrylic- or acrylate based thickener within a certain range exhibited an unexpected aesthetic appeal as well as advantageously remained in place on the skin, i.e. didn't drip or run from the surface on which it was applied.

Evaluation of Deposition of Skin Care Active Ingredient

The Inventive Examples were evaluated to measure the deposition of the skin care active ingredients onto the skin after being applied and rinsed off and compared to several commercially available products (Benchmark Product 1 and Benchmark Product 2)

The measurements were done with an analytical team. The Inventive Examples were applied to a person's forehead for 10 min, then rinsed off. Then, a few outer layers of skin were removed with tape stripping. The collected layers were analyzed with HPLC to quantify salicylic acid that was deposited on skin. The results are represented in Table 3 below.

TABLE 3

| | Inventive | Benchmark | Benchmark |
|---|---|---|---|
| Compositions | Example 1 | Product 1 | Product 2 |
| % of Salicylic Acid in | 1.5 | 2 | 2 |
| formulas | | | |
| Salicylic acid deposited (ug) | 47.71 | 37.21 | 35 |

The deposition of salicylic acid observed for the Inventive Ex.1 containing 1.5% of Salicylic Acid was 47.71 ug. The deposition of salicylic acid observed for the commercially available products containing 2% of salicylic acid was between 35 ug and 37.21 ug. The formulas containing 1.5% surprisingly exhibited a higher salicylic deposition compared to the commercially available products containing 2% of salicylic acid. Therefore, it appeared that the deposition of salicylic acid increased with the inventive examples. The combinations of a thickener such as a non-acrylate based rheology modifier with an acrylic- or acrylate based thickeners presented in the present disclosure surprisingly improved the deposition of salicylic acid.

While the disclosure has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A cleansing mask composition comprising:
a) from about 0.4 to 9 wt. % of a first thickener chosen from non-acrylate based rheology modifier;
b) from about 0.2 to about 2 wt. % of a second thickener chosen from acrylic- or acrylate based thickeners;
c) from about 2 to 25 wt. % of at least one surfactant;
d) a skin care active ingredient; and
wherein the composition has a loss modulus absolute value (G″) and a storage modulus absolute value (G′);
wherein the weight percentages are based on the total weight of the cleansing mask composition.

2. The composition of claim 1, wherein the loss modulus absolute value (G″) is less than three times the storage modulus absolute value (G′).

3. The composition of claim 1, wherein the composition spread easily on the skin.

4. The composition of claim 1, wherein the composition is drip-free.

5. The composition of claim 1, wherein the first thickener is chosen from steareth-100/PEG-136/HDI copolymer, PEG-240/HDI copolymer bis-decyltetradeceth-20 ether, Disteareth-100 IPDI (and) Steareth-100, PEG-120 methyl glucose trioleate, and combinations thereof.

6. The composition of claim 1, wherein the second thickener is chosen from Acrylates/C10-30 Alkyl Acrylates, Ammonium Polyacryloyldimethyl Taurate, Ammonium Acryloyldimthyltaurate/Vp Copolymer, Sodium Acryloyldimthyltaurate/Vp Copolymer and combinations thereof.

7. The composition of claim 1, wherein the first thickener is present from about 0.4 to about 2 wt. % based on the total weight of the cleansing mask composition.

8. The composition of claim 1, wherein the second thickener is present from about 0.2 to about 2 wt. % based on the total weight of the cleansing mask composition.

9. The composition of claim 1, wherein the at least one surfactant is chosen from the anionic surfactants, amphoteric surfactants and combinations thereof.

10. The composition of claim 1, wherein the at least one surfactant is present from about 5 to about 12 wt. % based on the total weight of the cleansing mask composition.

11. The composition of claim 1, wherein the skin care active ingredient is chosen from salicylic acid, alpha hydroxy acid, ceramides, ascorbic acid, antioxidants, vitamins, and combinations thereof.

12. The composition of claim 1, wherein the skin care active ingredient is deposited onto the skin.

13. The composition of claim 10, wherein the skin care active ingredient is salicylic acid.

14. A cleansing mask composition comprising:
a) From about 0.5 to about 8 wt % of a first thickener chosen from steareth-100/PEG-136/HDI copolymer, PEG-240/HDI copolymer bis-decyltetradeceth-20 ether, Disteareth-100 IPDI (and) Steareth-100, PEG-120 methyl glucose trioleate, and combinations thereof;
b) From about 0.2 to about 2 wt % of a second thickener chosen from Acrylates/C10-30 Alkyl Acrylates, Ammonium Polyacryloyldimethyl Taurate, Ammonium Acryloyldimthyltaurate/Vp Copolymer, Sodium Acryloyldimthyltaurate/Vp Copolymer and combinations thereof.
c) From about 2 to 25 wt. % of at least one surfactant;
d) From 0.05 to 15 wt. % of a skin care active ingredients, and
wherein the composition has a loss modulus absolute value (G″) and a storage modulus absolute value (G′);
wherein the loss modulus absolute value (G″) is less than three times the storage modulus absolute value (G′);
wherein the composition is drip free once applied on the skin;
wherein the weight percentages are based on the total weight of the cleansing mask composition.

15. A method of cleansing skin, comprising applying to skin the composition of claim 1.
